(19) United States
(12) Patent Application Publication
(10) Pub. No.: US 2015/0342894 A1
(43) Pub. Date: Dec. 3, 2015

(54) DRUG DELIVERY SYSTEMS AND RELATED METHODS OF USE

(71) Applicant: Textile-Based Delivery, Inc., Seattle, WA (US)

(72) Inventor: David Anderson, Rockport, MN (US)

(21) Appl. No.: 14/726,360

(22) Filed: May 29, 2015

Related U.S. Application Data
(60) Provisional application No. 62/005,772, filed on May 30, 2014.

(51) Int. Cl. A61K 9/50; A61K 31/137; A61K 31/4178; A61K 31/343

(52) U.S. Cl. CPC A61K 9/5021; A61K 31/343; A61K 31/137; A61K 31/4178; A61K 9/5073

(57) ABSTRACT

The present disclosure relates generally to drug delivery systems and related methods of use. The drug delivery systems can include one or more particles, each of which can include a biologically active compound dispersed therein. The drug delivery systems can also be configured to exhibit zero-order or near-zero-order release of the biologically active compound. The particles can be cylindrical or rod-like in shape, and can include a polymeric inner matrix having a biologically active compound dispersed therein, and a polymeric outer layer disposed at least partially around the inner matrix.

### CROSS-REFERENCE TO RELATED APPLICATIONS

This application claims the benefit of U.S. Provisional Application No. 62/005,772, filed May 30, 2014, titled “DEPOT DRUG DELIVERY SYSTEMS,” which is incorporated herein by reference in its entirety.

### TECHNICAL FIELD

The present disclosure relates generally to drug delivery systems and related methods of use. The drug delivery systems can include one or more particles, each of which can include a biologically active compound dispersed therein. The drug delivery systems can also be configured to exhibit zero-order or near-zero-order release of the biologically active compound.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view of a drug delivery system, according to an embodiment of the present disclosure.

FIG. 2 is a side view of the drug delivery system of FIG. 1.

FIG. 3 is an end view of the drug delivery system of FIG. 1.

FIG. 4 is another end view of the drug delivery system of FIG. 1.

FIG. 5 is a perspective view of a drug delivery system, according to another embodiment of the present disclosure.

FIG. 6 is a side view of the drug delivery system of FIG. 5.

FIG. 7 is a perspective view of a drug delivery system, according to yet another embodiment of the present disclosure.

FIG. 8 is a perspective view of a drug delivery system, according to still another embodiment of the present disclosure.

FIG. 9 is a side view of the drug delivery system of FIG. 8.

FIG. 10 is a graph illustrating the release profile of a drug delivery system, according to another embodiment of the present disclosure.

FIG. 11 is a graph illustrating the release profile of a drug delivery system, according to another embodiment of the present disclosure.

### DETAILED DESCRIPTION

The present disclosure relates generally to drug delivery systems and related methods of use. The drug delivery systems can include one or more particles, each of which can include a biologically active compound dispersed therein. The drug delivery systems can also be configured to exhibit zero-order or near-zero-order release of the biologically active compound.

For example, in some illustrative embodiments, the drug delivery systems disclosed herein include a biocompatible, non-erodible and/or non-biodegradable, injectable and/or implantable particle that is configured to exhibit a release profile which on a log-log plot of cumulative release of an active compound versus time has a slope greater than or equal to about 0.62, greater than or equal to about 0.75, or greater than or equal to about 0.87.

In another illustrative embodiment, the drug delivery systems include a biocompatible, non-erodible and/or non-biodegradable, injectable and/or implantable cylindrical or rod-shaped particle comprising a polymeric inner matrix having an active compound dispersed therein and a polymeric outer layer that is at least partially disposed around the inner matrix. The rod-like shape of the particles makes them better suited geometrically for a number of applications where, for example, deep penetration can be achieved beyond the point of injection, occlusion of tissue (e.g., corneal) can be minimized, or high surface areas can be attained but with good retention of particles at the site (for example, in a vaccine preparation), by virtue of the cylindrical or rod-like shape of the particles. In some of such embodiments, the polymeric outer layer can be substantially impermeable to the active compound, and the particle can conform to the following mathematical conditions: the ratio D/(Ku) is greater than 1, the ratio LK/D is less than 0.1, the aspect ratio L/d is between 1 and 50, where L is the length of the particle, d is the diameter of the inner matrix, D is the diffusion constant, and K is the dissolution constant of the active compound in the polymeric inner matrix, and u=1 centimeter (a standard unit of length). In further of such embodiments, the polymeric inner matrix can comprise one or more fluoroelastomers or fluorogreases.

In yet other illustrative embodiments, the drug delivery systems include a biocompatible, bio-erodible and/or biodegradable, injectable and/or implantable cylindrical or rod-shaped particle comprising a polymeric inner matrix having an active compound dispersed therein and a polymeric outer layer that is at least partially disposed around the inner matrix. In some of such embodiments, the polymeric outer layer can be substantially impermeable to the active compound, and the particle can conform to the following mathematical conditions: the ratio D/(Ku) is greater than 1, the ratio LK/D is less than 0.1, the aspect ratio L/d is between 1 and 50, where L is the length of the particle, d is the diameter of the inner matrix, D is the diffusion constant, and K is the dissolution constant of the active compound in the polymeric inner matrix, and u=1 centimeter (a standard unit of length). In further of such embodiments, the polymeric inner matrix can comprise one or more fluoroelastomers or fluorogreases.

For the purposes of promoting an understanding of the principles of the disclosure provided herein, reference will now be made to the embodiments illustrated in the drawings and specific language will be used to describe the same. It will be readily understood with the aid of the present disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. In some cases, well-known structures, materials, or operations are not shown or described in detail. While the various aspects of the embodiments are presented in drawings, the drawings are not necessarily drawn to scale unless specifically indicated.

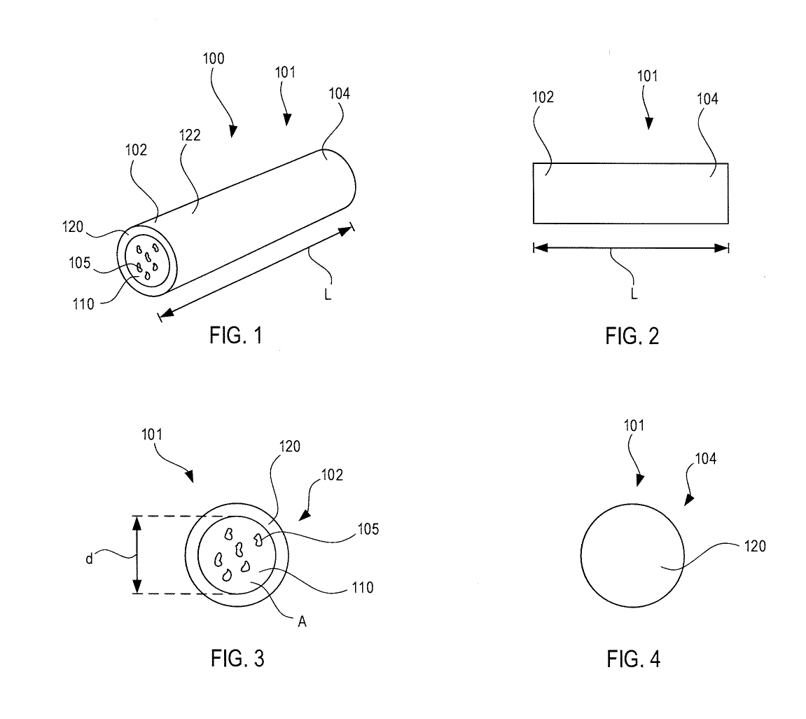

FIGS. 1-4 depict a drug delivery system 100 according to an embodiment of the present disclosure, where FIG. 1 is a perspective view of the drug delivery system 100, FIG. 2 is a side view of the drug delivery system 100, FIG. 3 is an end view of the drug delivery system 100, and FIG. 4 is another end view of the drug delivery system 100. As shown therein, the drug delivery system 100 can include one or more particles 101, each of which can include a biologically active compound 105. The biologically active compound 105 can also be referred to as a biologically active ingredient, an active compound, active ingredient, or a drug. In certain embodiments, the biologically active compound 105 may be in the form of a fine powder formulation, which may contain one or more excipients or complexing agents.

Various types of active compounds 105 can be used with the particles 101 disclosed herein, including, but not limited to, drugs, nutrients, hormones, chemotherapeutics, antibiotics, growth inhibitors, growth factors, etc. Illustrative active compounds 105 also include anti-abuse or “replacement therapy” drugs (e.g., buprenorphine, naloxone, etc.), local anesthetics (e.g., at an arthritic joint), opioids (e.g., fentanyl, carfentanil, etc.), opiates, steroids (e.g., testosterone, estrogen, progesterone, corticosteroids, glucocorticoids, dexamethasone, mineralcorticoids, Vitamin D, progestins, contraceptives), peptide hormones (e.g., insulin in slow-release form), insulin sensitizers (e.g., growth factors (e.g., bone morphogenic protein, vascular endothelial growth factor (“VEGF”), etc.)), multiple sclerosis related drugs (e.g., fingolimod, interferons, etc.) anticancer drugs, statins (e.g., rosuvastatin), tumor necrosis factor (“TNF”) inhibitors, cannabinoids (e.g., for fibromyalgia and glaucoma), migraine related drugs (e.g., almotriptan, naratriptan, etc.), vasodilators (e.g., fenoldopam), anticonvulsants (e.g., benzodiazepines), weight loss agents (e.g., phentermine, lorcaserin), gastrointestinal (“GI”) tract drugs (e.g., bowel antispasmotics), cardiac drugs, anti-HIV drugs (e.g., rilpivirine), psychiatric drugs or other drugs that are plagued by poor patient compliance (e.g., drugs for treating bipolar disease of schizophrenia), and drugs for the youth and elderly (e.g., drugs for Alzheimer's disease).

Other types of active compounds can also be used. For example, in certain embodiments, such as ophthalmic applications where the particle 101 is configured to be implanted in or around the eye, the particle 101 can be used to deliver a systemic drug and/or an ophthalmic related drug, such as a steroid, antifungal, epinephrine, beta-blocker, miotic, prostaglandin, or nutrient such as Vitamin A or carotene, etc. If a rod-shaped particle of the disclosure is inserted in a direction normal to the tangent plane of the eye (more simply, “straight into the eye”), then the effective area on the sphere of the eye taken up by the particle can be very small; this contrasts sharply with the case of a layered (flat) material. Glaucoma related drugs can also be used, including latanoprost, echothiophate, brimonidine, and demecarium, etc. In further embodiments, such as anti-cancer applications for example, the particle 101 can also be used as follow-on treatment after a higher-dose therapy, so as to provide a long-term release of an active compound 105 for continued treatment and/or protection. In certain embodiments, the particle 101 can be used alone or in combination with other treatment options, including but not limited to surgery (e.g., lumpectomy, mastectomy, sentinel node biopsy, axillary lymph node dissection, cryosurgery, etc), radiation therapy, chemotherapy, hormone therapy, and the like.

In some embodiments, element 105 in the Figures referred to herein as “active compound” is a solid. Element 105 is to be viewed in the context of this paragraph as an active grain that is either added as a pharmaceutically acceptable powder comprising biologically active compound(s), or precipitated within the inner matrix. Such a grain can be a solid having a crystalline or polycrystalline form. In certain embodiments, a plurality of individual active compounds may be arranged to form a polycrystalline structure. In other embodiments, the active compound might be a liquid at ambient temperature, and in some of such cases, co-crystals of the active compound can be prepared and dispersed or otherwise loaded into the inner matrix 110 in a manner that is analogous to dispersing or loading a solid crystal (e.g., small molecule drug crystal). For example, a number of compounds can be used to form solid complexes with liquids and other amorphous materials; cyclodextrins for example form inclusion compounds with many liquids, urea forms complexes with straight-chain molecules, etc. It will thus be appreciated that, in some embodiments, reference numeral 105 in the illustrated embodiment can be used to describe dispersed active or active-containing crystals. In further embodiments, reference numeral 105 can be used to represent amorphous solid or solid-like active compounds in dispersed form, a solid dispersion. In still further embodiments, reference numeral 105 can represent a co-crystal of active compound.

The amount of active compound 105 dispersed within the particle 101 can vary as desired. In certain embodiments, the active compound 105 present in the particle 101 is in an amount suited for delivering the active compound 105 to subjects in sufficient amounts to achieve a desired therapeutic effect. For example, in some embodiments, the particle 101 includes less than about 10 g, less than about 1 g, or less than about 100 mg of active compound 105. Other amounts can also be used, for example, depending on the type of active compound 105 that is used and the treatment or dosage regimen that is desired. The amount of active compound 105 dispersed within the particle 101 can also vary depending on the age, gender, or weight of the intended recipient. In some applications, the daily delivered dose may be considerably lower than doses characteristic of short-term or acute care, such as for example when a small dose of anticancer agent, say, or growth hormone is useful in maintaining remission from a disease or disorder, on a home-based or outpatient regimen.

The drug delivery systems 100 disclosed herein can also be referred to as depot drug delivery systems. For example, the drug delivery systems 100 can store an amount of active compound 105 (which can be dispersed in the inner matrix 110), and release it over time at a substantially constant rate. For example, release of the active compound 105 can occur over a period of minutes, hours, days, months, etc. For example, in an illustrative embodiment, a particle 101 including 100 mg of active compound 105 can be configured to release the active compound 105 over a period of about 1 month. In such embodiments, the daily release of the active compound 105 from the particle 101 can be about 3 mg. As can be appreciated, the release rate and the concentration can also be varied as desired, e.g., according to the treatment or dosage regimen desired, the strength of the active compound 105, and/or the age, gender, or weight of the intended recipient.

With continued reference to FIGS. 1-4, in some embodiments, the particle 101 comprises an elongate or longitudinally elongated shape or structure. The particle 101 can also include a first end portion 102 and a second end portion 104. In some of such embodiments, the particles 101 can be substantially cylindrical or rod-like in shape. Substantially cylindrical or rod-like shaped particles 101 can also be described as fibers. Further, in some embodiments, the particle 101 can be formed by cutting a longitudinal section of an elongated fiber. In some of such embodiments, reduced temperatures can be used to reduce the elastic behavior of the fiber components, aiding in cutting, molding, shaping, and/or forming the particle 101. Other shapes are also contemplated, including, but not limited to, spherical shapes, ellipsoid shapes, etc.

As further shown in FIGS. 1-4, the particle 101 includes an inner matrix 110, which can also be described as a core layer, an inner layer, or a first layer. The inner matrix 110 can extend longitudinally and continuously for approximately the length of the particle 101 (e.g., from the first end portion 102 to the second end portion 104). The particle 101 also includes an outer layer 120, which can also be described as a coating, a coating layer, a skin, or a second layer. As shown in the illustrated embodiment, the outer layer 120 can be at least partially disposed around the inner matrix 110. In some embodiments, such an arrangement can be described as a nested structure. Additional layers can also be used. For example, one or more layers (e.g., intermediate layers) can be disposed between the inner matrix 110 and the outer layer 120. Further, one or more additional layers can also be disposed on the exterior surface 122 of the outer layer 120. For example, one or more additional layers can be disposed on the exterior surface 122 of the outer layer 120 to modify or increase the biocompatibility of the particle 101.

In some embodiments, the inner matrix 110 and the outer layer 120 comprise separate and distinct polymeric materials. The materials of the inner matrix 110 and the outer layer 120 can also be different, and can be configured to exhibit different properties. For example, the inner matrix 110 can comprise a first material that is configured to host or otherwise contain the active compound 105 such that the active compound 105 can be dispersed therein. The first material of the inner matrix 110 can also allow the dissolution of the active compound 105 into the inner matrix 110, which can occur at a relatively slow rate as the mathematical condition D/Ku>1 can place a relatively low upper limit on the dissolution rate constant K. Further, in some embodiments, the particles 101 can be desired to release the active compound 105 over many days or even months. In some such embodiments, the inner matrix 110 allows for both dissolution of the active compound into the inner matrix 110 (e.g., from the dispersed solid biologically active compounds or crystals 105), and diffusion of the dissolved active compound molecules from the inner matrix 110 (e.g., via the exit or open end of the particle 101). One skilled in the art will also recognize that any given active compound molecule might in fact dissolve and/or recrystallize many times within the inner matrix 110, on the same and/or different crystals; however, in the mathematical analyses herein and those referred to, this is typically accounted for in the definition of the rate constants, which reflect the overall process of dissolution into the elastomeric domains and diffusional flow into, and eventually exiting from, the uncoated exit regions.

As further detailed below, in some embodiments, the inner matrix 110 comprises an elastomeric polymer matrix. However, the inner matrix 110 need not be 100% elastomeric; rather, it can include one or more glassy and/or crystalline polymer domains. In such embodiments, the elastomeric domains of the inner matrix 110 can form a continuous, material-spanning bulk or network, which allows a molecule of the active compound to diffuse from one end of the particle 101 to the exit region without ever having to leave the elastomeric network (e.g., from end 104 to end 102 of the particle 101 of the illustrated embodiment).

Various materials, including biocompatible polymeric materials, can be used in or as the inner matrix 110, allowing for dissolution of the dispersed solid 105 and diffusion of the active compound. For example, the inner matrix 110 can comprise one or more polymers or copolymers of fluoroelastomers, fluorogreases, polysiloxanes (silicones), acetoxysilicone, polyurethanes, polyanhydrides, polyisobutylene, elastin, natural rubber (polyisoprene), chloroprene, neoprene, butyl rubber, styrene-butadiene rubber (“SBR”), nitrile rubber, epichlorohydrin rubber, polyether block amides, ethylene-vinyl acetate (“EVA”), acrylics, siliconized acrylics, copolymers such as poly(styrene-b-isobutylene-b-styrene), acrylonitrile butadiene styrene (“ABS”), and derivatives and mixtures thereof. Thermoplastic elastomers, such as fluoropolymers (e.g., Viton®), polyolefin blends (TPE-o), elastomeric alloys (TPE-v or TPV, such as Forprene), thermoplastic polyurethanes (“TPU”), thermoplastic copolyesters, poly(methyl methacrylate) (“PMMA”) polyisoprene block copolymers, thermoplastic polyamides, and derivatives and mixtures thereof can also be used, including Arnitel® (DSM), Solprene® (Dynasol), Engage® (Dow Chemical), Hytrel® (Du Pont), Dryflex® and Mediprene® (ELASTO), Kraton® (Kraton Polymers), and Pibiflex®. The materials can be crosslinked, or non-crosslinked, as desired. In some embodiments, an elastomer can refer to a low-crystalline, non-glassy, and/or crosslinked polymer. Crosslinking of the inner matrix 110 can reduce tackiness, aid in securing the solid active compound 105 substantially in place (as can be described by the term “immobilized”), and can limit or prevent loss of matrix material during production, use, and laundering. In some embodiments, the crosslinking can be sufficient to yield what is known in the art as “infinite molecular weight.” The high molecular weight of a polymer, particularly when crosslinked, can also mitigate migration or leakage of the material from the inner matrix 110, and yet the low-crystallinity and non-glassy characteristic (i.e., the glass transition temperature Tg being below body temperature) of the elastomeric domains of the inner matrix 110 material can allow for dissolution and subsequent diffusion of an active compound 105 (e.g., a solid active compound) initially dispersed within the polymer inner matrix 110. With this functional definition, certain materials will fall under this definition even though they are typically referred to by other terms, such as “fluoropolymer grease”, or “release agent”, or “caulk”, or “rubber”, or “sealant”, or “fluorogrease”, or “damping fluid”, etc.

In certain embodiments, the inner matrix 110 comprises various water-soluble polymers, including polyhydroxyethyl methacrylate (“PolyHEMA”), gelatin, starch (including derivatives thereof), polyethylene glycol, celluloses, natural gums such as gum arabic, gum tragacanth, xanthan gum, guar gum, gellan gum, dextran, or derivatives and mixtures thereof. The water-soluble polymers can be crosslinked, or non-crosslinked. The water-soluble polymers can also be hydrated. For example, in some embodiments, the inner matrix 110 comprises a crosslinked polymer material that can be hydrated to equilibrium swelling, such that the D and K parameters (further discussed below) can be approximated by the corresponding values in water. Further, in some instances, such as where faster release rates are desired, a non-volatile and non-toxic solvent (e.g., liquid) such as tocopherol can be used to swell the inner matrix material.

Further, in some embodiments, the disclosed drug delivery systems 100 can be substantially non-erodible, or substantially non-biodegradable. In other embodiments, the disclosed drug-delivery systems 100 are substantially bioerodible, or substantially biodegradable. Non-erodible or non-biodegradable drug delivery systems 100 can be formed in various ways. For example, in some embodiments, the inner matrix 110 of the particle 101 can include elastomers that are non-glassy and have low-crystallinity domains. In certain embodiments, hydrophobic elastomers that exclude or do not readily absorb water can also be used. Further, in some embodiments, crosslinked polymers can be used that inhibit leakage of the polymer into the surrounding environment and/or inhibit entry of compounds including high-molecular weight compounds) such as proteins, lipoproteins, and high-molecular weight polysaccharides.

In some embodiments, the inner matrix 110 includes a non-fluorinated polymer, such as for example a polysiloxane, but is covered at the exposed regions (typically one or both ends 102, 104) with a fluoropolymer, fluorogrease, or fluoro coating. Such an arrangement can provide added flexibility in choosing the inner matrix 110 material, e.g., for dissolution rate and cost optimizations, while at the same time applying the fluoropolymers' ability to prevent or limit water and oils from entering the inner matrix 110. Such an arrangement can also reduce or minimize the amount of fluoropolymer that is used, which can be important from a regulatory and/or toxicity standpoint.

As previously mentioned, in some embodiments, the inner matrix 110 is crosslinked. For example, physical crosslinking can be obtained by the use of block copolymers containing crystalline or glassy domains. Illustrative block polymers that can be used for physical crosslinking, include, but are not limited to, poly(methyl methacrylate) (“PMMA”), polytetrafluoroethylene (“PTFE”), biocompatibility-enhanced fluoropolymers, and hard polyurethane segments. Such block polymers can also allow for extrusion-based processing. Inert atmospheres can also be used during certain stages of processing when temperatures on the order of 200 degrees Celsius might occur as transients.

In certain embodiments, a block copolymer of well-controlled block structure forming a “hexagonal” or “cylinder” phase morphology, in which one of the blocks is an elastomer, can be used as the inner matrix 110, provided it is cut in the direction of alignment of the cylinders. The “hexagonal phase” (also called “cylindrical morphology”) microstructure, featuring long cylinders of one block packed on a hexagonal lattice inside a continuum of another block with which the first block is immiscible), is described in detail in U.S. Pat. No. 6,638,612 to Anderson. In such embodiments, if the elastomeric blocks make up the “cylinders”, and a hydrophilic block makes up the continuum between cylinders, it can be effective at impeding ingress of both aqueous and oily liquids.

In certain embodiments, elastomers for the inner matrix 110 are fluoroelastomers, which can repel both aqueous and oily materials. For example, the inner matrix 110 can comprise fluoroelastomers having greater than about 30%, greater than about 40%, greater than about 50%, greater than about 60%, or greater than about 70% fluorine substitution. Illustrative fluoroelastomers that can be used include, for example, Viton® (DuPont), FPM, FKM, Dai-El® (Daikin Chemical), Dyneon® (3M), Elaftor®, Technoflon®, and others. In certain embodiments, cold temperatures can cause the elastomer-containing particle 101 to become brittle, which can lead to shattering, and if shipping in cold climates is anticipated, a more cold-resistant fluoroelastomer could be used, such as the GFLT grade of Viton®.

As can be appreciated, in certain embodiments, polymers can be desirable as the inner matrix 110 for a given active compound 105 when the combined polymer-active compound mixture: 1) satisfies the conditions given herein for dissolution-limited release; 2) is elastomeric or a high-viscosity liquid at the application temperature, normally of about 35-38 degrees Celsius; 3) is very low in extractables and shedded material, except for the active compound 105 itself; 4) is repellant against the imbibition, over the lifetime of the application, of liquids and lipids that can significantly affect the release profile, such that, for example, the slope of a log-log plot of the cumulative release versus time is not greater than about 0.75; and 5) does not cause toxic, allergic, nor autoimmune responses that are so severe as to preclude the application. In addition, in certain embodiments, any implanted materials should be readily removable by a physician without extraordinary measures, or can be reasonably expected to either be removed by bodily processes, or be well tolerated by the body at the implant site for long periods of time relative to the progression of invasive and non-invasive procedures associated with the condition that is treated with the application of an implant of the present disclosure.

In the case of elastomers that contain organic solvents (e.g., such as methylethyl ketone in the case of Viton® (Dupont)) to liquefy the elastomer, where the processing temperatures required by the elastomer are not elevated, then the issues associated with flashing of solvents and resulting compositional and physical changes can be addressed through methods known to one skilled in the art. In the case of elastomers that do not rely on organic solvents for viscosity reduction, then the normal means of processing is heating to an elevated processing temperature, performing mixing (with the active compound) and extrusion or other melt-processing step. In some embodiments, this can require accelerated cooling and/or processing in a reduced-oxygen environment, in order to limit chemical degradation of the active compound 105.

A powder of the active compound 105 can be prepared either prior to physical mixing with the inner matrix 110 material (or inner polymer matrix) in a liquefied state, or in situ as the active compound 105 crystallizes out inside the inner matrix 110 due to cooling and/or evaporation of solvent, and control of crystallite size can affect certain aspects of the release profile, with crystallite size of about 100 microns or less, about 20 microns or less, or about 5 microns or less. For the first approach, methods for producing small crystals of an active compound can be categorized according to whether larger starting materials are milled down to smaller size (the “top-down” approach), or microscopic crystals are engineered from the start (the “bottom-up” approach). Methods for milling include, but are not limited to, high-shear homogenization, high-pressure homogenization (also known as microfluidization), ultrasonication, wet milling, ball milling, and others. “Bottom-up” methods can rely on precipitation or crystallization in the presence of size-reductive methods such as homogenization and sonication; alternatively, actives can be crystallized within microstructures, such as emulsion droplets, liposomes, microparticles, etc., that can limit the size of the resulting crystals.

In the second approach where the active compound 105 crystallizes out during cooling and/or evaporation of solvent, crystallite size can be adjusted by control of nucleation conditions as is known in the art. Such methods of control include, for example: rate of cooling; rate of evaporation; presence of nucleating material; and application of strong shear during evaporation.

In some embodiments where the materials (e.g., the inner matrix 110) of the particle 101 is erodible or biodegradable, at least in the coated regions the rate of erosion of the erodible polymer is expected to be much slower than if the same polymer were exposed “naked” to the same environmental conditions. If the outer, coating polymer is erodible, then if significant erosion (e.g., greater than about 10%) of the coating occurred over a timescale that is comparable to the desired timescale of active compound release, then this would change the release profile as compared to the near-zero-order kinetics described herein, and this effect would have to be accounted for.

Illustrative erodible or biodegradable polymers include, but are not limited to, poly-lactic acid, poly-L-lactide, poly-glycolic acid and their copolymers as well as other polyesters, polycaprolactone, biopolymers such as based on collagen or gelatin or other peptide, certain natural gums, certain polysaccharides, chitosan and derivatives, and derivatives and mixtures thereof. Other known erodible or biodegradable polymers in the field of drug delivery can also be used.

The outer layer 120 can be at least partially disposed around and/or over the inner matrix 110. For example, in some embodiments, the outer layer 120 is around or disposed over between about 80% and about 99.9%, between about 85% and about 99.5%, or between about 90% and about 99% of the volume of the inner matrix 110. In further embodiments, the inner matrix 110 is covered by the outer layer 120 everywhere except at one or more ends 102, 104 of the particle 101, thereby confining release of the active compound 105 to a relatively small area and, thus, allowing for extended release of the active compound 105 over extended periods of time (e.g., days, months, etc.). And in certain embodiments, such as the illustrated embodiment, the outer layer 120 is disposed around the inner matrix 110 such that only a first end portion 102 of the particle 101 is uncovered. For example, FIG. 3 depicts an end view of the particle 101 illustrating the first end portion 102 not being covered by the outer layer 120, and FIG. 4 depicts an end view of the particle 101 illustrating the second end portion 104 being covered by the outer layer 120. In other embodiments, both end portions 102, 104 can be uncovered. As can be appreciated, an uncovered portion of the inner matrix 110 can also be described as a non-occluded portion, or a portion that is free of the outer layer 120. Analogously, the covered portions of the inner matrix 110 can be described as occluded portions.

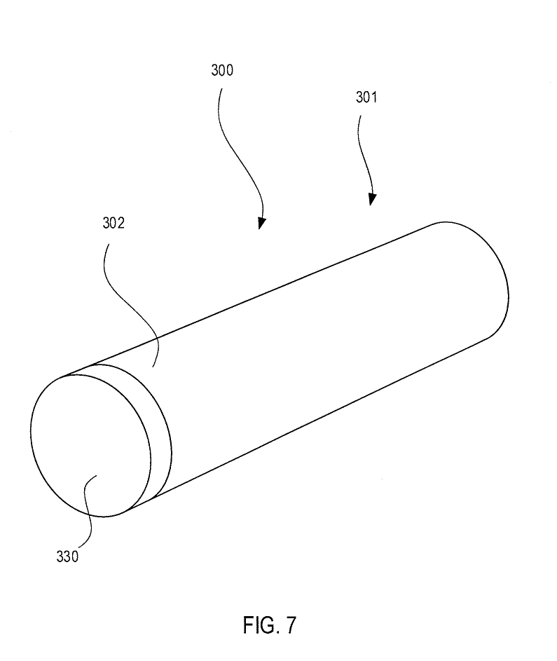

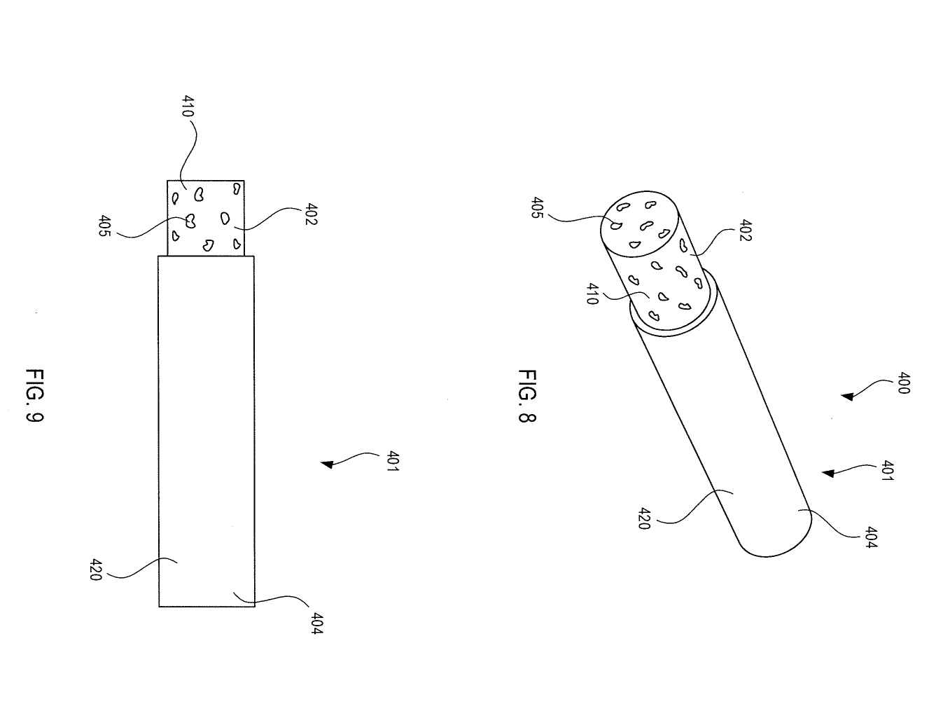

In some embodiments, the ingress of oily or lipidic materials into the inner matrix 110 over time (e.g., into the uncovered portion of the inner matrix 110), which could affect the release profile of the particle 101 can be prevented or limited by one or more of the following: 1) the outer layer 120, 2) a semipermeable membrane or other material covering the uncovered portion of the inner matrix 110 (e.g., such as is discussed with reference to FIG. 7), or 3) the presence of fluoropolymers in the inner matrix 110 (e.g., which can limit the ingress of oily or lipidic materials). In some embodiments, it may be desirous that ingress of oily and lipidic materials is such that when particles 101 are immersed in a test fluid containing oils and lipids (e.g., whole milk), the uptake over one month in total oils and lipids is less than about 10%, less than about 5%, less than about 3%, less than about 2%, less than about 1% or less than about 0.5%, less than about 0.25%, or less than about 0.1% of the weight of the inner matrix material 110.

In certain embodiments, ingress of oily and lipidic components can be limited by including thiolene-based elastomers, or fluoroelastomers such as fluorinated norbornene elastomers, perfluoropolyether elastomers, tetrafluoroethylene propylene copolymer and terpolymer, FKM and FFKM fluoroelastomers (as defined by ASTM D1418 standard), and derivatives and mixtures thereof. Use of such materials can yield an inner matrix 110 that can substantially exclude both hydrophilic and hydrophobic liquids from ingress and from interfering with the release kinetics of the particle 101. In certain embodiments, for example, the inner matrix 110 comprises a fluoropolymer “release agent” that includes one of the two main ingredients in the Scotchpak Liner 1022 and related liners from Minnesota Mining and Manufacturing (“3M”).

The outer layer 120 can comprise a material that can be impermeable, or substantially impermeable, to the material of the inner matrix 110 and/or the active compound 105. In such embodiments, dissolution or release of the active compound 105 from the particle 101 can be limited to, or substantially limited to, regions of the inner matrix 110 that are not covered by the outer layer 120. Such a configuration can allow for dissolution limiting release of the active compound 105 by the particle 101.

Various materials can be used in the outer layer 120, including polymeric materials. In some embodiments, the materials of the outer layer 120 can be biocompatible, safe, non-immunogenic or low-immunogenic, and/or hypoallergenic. Surface treatments (e.g., coatings) can also be applied to the outer layer 120 and/or the particle 101 so as to improve biocompatibility, including, but not limited to, coatings such as polyethyleneglycol (“PEG”) chains, collagen, phospholipid, polysaccharide, proteinaceous material such as albumin, or specialized coatings such as the Carmeda® coating developed from heparin, or covalently-bonded phospholipids or fragments of phospholipids.

Illustrative materials that can be used for the outer layer 120 include, but are not limited to, polymers and copolymers of polypropylene, polyvinyl chloride, polytetrafluoroethylene (“PTFE”) (e.g., non-porous PTFE), polyvinylidene fluoride (“PVDF”), PMMA, polycarbonate (e.g., Lexan), polybutylene terephthalate, polyethylene terephthalate (“PET”), high-density polyethylene, polyamide (e.g., nylon), polyimide, celluloid, phenol-formaldehyde resin, and polystyrene and derivatives and mixtures thereof. Polylactide (“PLA”) can also be used in some embodiments where the in vivo degradation rate of the PLA is relatively slow compared to the release of active compound 105, or slower than the release rate of the active compound 105.

Other materials can also be used in the outer layer 120, including materials having a low solubility and/or low permeability, highly crystalline polymers, or polymers that are in the glassy state at or near ambient temperatures. In some embodiments, particles having a melting temperature low enough to allow easy processing can also be used.

As further shown in FIGS. 1-4, in some embodiments, the particle 101 is void of moving parts, such as mechanically moveable parts (e.g., pistons, etc.). In other words, moving parts are not present and/or need not be required to achieve the desired release of the active compound 105. Rather, the particle 101 can be described as being static, or void of mechanically moveable parts.

Electrical and electroosmotic currents are also not required for the release of the active compound 105. And, in some embodiments, nanoporous membranes are not needed or included. In further embodiments, organic small-molecule liquids are also not required, which can be disfavored because of their propensity to disperse at a rate that is difficult to control upon extended contact with aqueous bodily fluids (e.g., such as those encountered in the subcutaneous space). Further, in some embodiments, organic solvents are not used, which also can be advantageous. Benzyl alcohol, for example, can cause allergy-related reactions, hemolysis, and other side effects that can be attributed to organic solvents.

In some embodiments, the particle 101 also does not require electrical, magnetic, flow or electroendoosmotic fields in order to achieve highly uniform release as a function of time over periods of weeks or months, and furthermore the particle 101 and drug delivery systems 100 disclosed herein can be largely independent of such fields. Such particles 101 and drug delivery systems 100 can be advantageous as such fields can depend on the ionic strength of the environment, which can be difficult to control. In some of such embodiments, the release is not dependent upon release-controlling ionic interactions.

In some embodiments, the particle 101 is biocompatible and suitable for injection or implantation into the body of a mammal, such as a medical patient. For example, in certain embodiments, the particle 101 can be suitable for subcutaneous, intramuscular, intradermal, and/or intraocular injection or implantation. The particle 101 can also be deposited into the body of a mammal in other ways, including by irrigation methods (e.g., irrigation of one or more particles 101 in a solution), insertion methods, or by other known methods for depositing compositions, particles and/or powders into the body of a mammal.

A wide variety of injection and/or implantation methods can be used in accordance with present disclosure, including injection and/or implantation methods for single particles 101 and pluralities of particles 101. For example, a single particle 101 (e.g., rod-shaped particle), or alternatively a plurality of particles 101, can be situated within bodily tissue in a manner that places the “open” areas of release in regions where targeting of the active compound makes sense therapeutically. For example, a particle 101 (e.g., rod-shaped particle) comprising a therapeutic dose of an ophthalmic drug such as a steroid, antibiotic/antimicrobial, pilocarpine, statin, local anesthetic, vitamin, etc., could be implanted by direct insertion of one or more such particles into the body of the eye with proper attention paid to the exact location of the open release regions, which can in many cases be selected so as to be more effective in these specific locations. For delivery to the back of the eye, which can be difficult to target, particles (e.g., rod-shaped particles) produced with one of the two ends open and the other end closed could be implanted into the eye open end first; whereas for delivery of drugs to the front of the eye (cornea, tear film, etc.) one could implant a particle 101 with the open portion more forward in the eye. For delivery of an active compound into tissue below the surface of the skin, one could insert particles (e.g., rod-shaped particles) open end downward, where the length of the particles are sufficient to reach the targeted depth. Injection or implantation of an aqueous dispersion of particles (e.g., rod-shaped particles) through a very fine-bore needle (e.g., a 22 gauge or thinner needle) can be used to orient the particles (e.g., rod-shaped particles) in tissue. Larger particles (e.g., rod-shaped particles) stiff enough to maintain shape during insertion can also be inserted directly into certain target tissues.

As previously discussed, in particular embodiments, the particle 101 can be suitable for subcutaneous, intramuscular, intradermal, and/or intraocular injection or implantation. Other routes of administration are also contemplated, including auricular (otic), buccal conjunctival, cutaneous, dental endocervical, endosinusial, enteral epidural, interstitial, intra-abdominal, intra-amniotic, intra-articular, intracardiac, intracartilaginous, intracaudal, intracavernous, intracavitary, intracisternal, intracorneal, intradermal, intradiscal, intraductal, intradural, intraepidermal, intraesophageal, intragingival, intralesional, intraluminal, intralymphatic, intramuscular, intraocular, intraovarian, intrapericardial, intraperitoneal, intrapleural, intraprostatic, intrasinal, intraspinal, intratesticular, intrathecal, intrathoracic, intratubular, intratumor, intrauterine, intravesical, intravitreal, laryngeal nasal, ophthalmic, percutaneous, periarticular, peridural, perineural, periodontal, respiratory, retrobulbar, soft tissue, subarachnoid, subconjunctival, topical, transdermal, transplacental, transtracheal, urethral, and vaginal related administration methods.

In certain embodiments, the particle 101 is configured to be used in the treatment of cancer, such as bone and/or breast cancer. In some of such embodiments, the particle 101 can be deposited (e.g., injected, implanted, etc.) into a bone of the mammal (e.g., for the treatment of bone cancer), and into breast tissue (e.g., for the treatment of breast cancer). In further embodiments, the particle 101 is configured to be deposited (e.g., injected, implanted, etc.) into or adjacent to a tumor. In some of such embodiments, the particle 101, can be configured to release an active compound 105, such as an anticancer or antiproliferative drug, to the tumor at a substantially constant rate over time.

Further, in some embodiments, the particle 101 is configured to be explanted or otherwise removed from the subject or recipient (e.g., medical patient) after use. For example, the particle 101 can be explanted after delivering a desired quantity of active compound 105. The particle 101 can also be explanted after exhaustion of the active compound 105, or after the active compound 105 has been substantially or completely released from the particle 101. If desired, the particle 101 can also be explanted after partial release of the active compound 105. Further, in certain embodiments (e.g., embodiments wherein the particle 100 is non-erodible) the particle 101 is, at the time of implantation, intended to be explanted unless the recipient (e.g., medical patient) dies before the explantation is to occur.

In some embodiments, a high-modulus outer layer 120 over the inner matrix 110 can aid in implantation and/or explantation. For example, regardless of how soft and friable the inner matrix 110 is, during removal the entire particle 101 can be removed as a single unit due to the encapsulating effect of the outer layer 120. In further embodiments, the outer layer 120 can be interior to, or supplanted by, a solid tubing of metal, ceramic, plastic, or other advanced material.

In other embodiments, the particle 101 is not configured to be removed, but is instead configured to remain within the recipient (e.g., medical patient). For example, a particle 101 implanted in an tumor (e.g., and intratumor particle) could be left in the tumor indefinitely, or at least until the particle 101 is degraded or otherwise eliminated by the body. In some of such embodiments, the presence of the particle 101 is negligible when compared to the tumor that is being targeted by the particle 101.

In some embodiments, including embodiments where the particle 101 is not intended to be explanted, the particle 101 can comprise biocompatible materials, and materials (e.g., collagen) that promote the ingrowth of tissue on the particle 101. Use of such materials in the outer layer 120, or exterior to the outer layer 120, can yield particles 101 that need not be removed by explantation.

In some embodiments, the release of active compound or drug from the particles depends characteristically on 3 steps, which recognizes that a relatively high fraction of the active compound or drug in the particle (except perhaps for the terminal portion of the release period) is in one of the solid drug crystals, co-crystals, or amorphous dispersed solids, and these steps occur with certain chronological constraints: A) dissolution of active compound from the dispersed crystals; B) diffusion of molecules of active compound away from crystal surfaces to the exposed surface areas at the exit or open regions; and C) diffusion of the active compound out of the particle into body tissue or fluid through these exit or open regions. The first two steps can happen in a back and forth manner, as dissolved active compound or drug can re-crystallize back onto the same or a different crystal. Nevertheless, this does not change the fundamental fact that either diffusion or dissolution can be rate-limiting.

The particle 101 can be configured to yield dissolution limiting release of the active compound 105. The particle 101 can also be configured to exhibit zero-order or near-zero-order release of the biologically active compound 105. Zero-order kinetics or zero-order release kinetics refers to a release rate of an active compound 105 in which the rate of release is independent of the amount of active compound 105 remaining, that is, to the zero power of the amount of active compound 105 remaining. The cumulative release profile of zero-order release kinetics has a constant slope (equal to the release rate), which on a log-log plot has a slope of 1. Dissolution-limited release, is the term applied when the rate-limiting or slowest process in the chain of events leading to the movement of the active compound to the open (not occluded by the outer layer) regions for release into body tissue or fluid is the dissolution of the active compound 105 into the inner matrix 110, and in the present disclosure this yields an approximately zero-order release profile.

Near-zero-order release or near-zero kinetics refers to a release profile that, for the majority of the release profile (e.g., greater than 50%, greater than 60%, greater than 70%, greater than 80%, greater than 90%, or greater than 95%), the release of the active compound 105 is limited by the dissolution of the remaining undissolved active compound 105, which under the conditions described herein can lead to zero-order release. In some embodiments, a least-squares fit to a log-log plot of cumulative amount of active compound 105 released versus time will have a slope close to about 1.0, between about 0.75 and about 1.33, between about 0.825 and about 1.15, or between about 0.9 and about 1.11 for a near-zero-order release profile.

In certain embodiments, the particle 101 is configured to exhibit release profiles which on a log-log plot of cumulative release of active compound 105 versus time have a slope greater than or equal to about 0.62, greater than or equal to about 0.75, or greater than or equal to about 0.87.

In some embodiments, the zero-order or near-zero-order release can be attributed at least in part to the polymeric or polymer-based materials used in the inner matrix 110 and outer layer 120 of the particle 101. In such embodiments, the disclosed drug delivery systems 100 can exhibit zero-order or near-zero-order release kinetics with materials that are organic, substantially non-toxic (or low in toxicity), inexpensive, commonly available, easily functionalized, environmentally degradable, and pharmaceutically acceptable for injectable and/or implantable routes of administration.

In certain embodiments, the release properties of the particle 101 can be defined by, conform to, or substantially conform to the following equations:

$$Release rate = Q = \frac{d M}{d t} = A {C}_{S} \sqrt{DK}$$

$$Total mass of drug released = A {C}_{0} L$$

$$Duration of release = \frac{Total drug released}{Release rate} = A {C}_{0} L / ( A {C}_{S} \sqrt{DK} = ( {C}_{0} / {C}_{S} ) L / \sqrt{DK}$$

Where A is the area of the inner matrix 110 not covered by the outer layer 120 (e.g., the surface area shown in FIG. 3), L is the length of the particle 101, D is the diffusion rate of the active compound 105 in the inner matrix 110, K is the dissolution constant of the active compound 105 in the inner matrix 110, C₀ is the initial concentration of active compound 105 in the inner matrix 110 (including dissolved and undissolved active compound 105), and C_{S} is the saturation concentration of the active compound 105 in the inner matrix 110.

As demonstrated above, in some embodiments, the release rate can be independent of the length L of the particle 101. In contrast, the duration of the release can depend upon the length L of the particle 101. As such, the duration of the release can be controlled by adjusting the length L of the particle 101, without affecting the rate of release. The drug delivery systems 100 disclosed herein can therefore provide not only for near-constant drug release, but also for independent control of release rate and duration of release. This can be advantageous as the choice of the material (e.g., polymeric material) that forms the inner matrix 110 and/or outer layer 120 can be selected based on factors other than D and K, such as cost, ductility, processability, crosslinking considerations, tack/adhesion, etc. Further, as can be appreciated, one may not want to be restricted in polymer selection in order to meet certain kinetics requirements (D and K) without an easily adjustable parameter such as the aspect ratio of the particle 101.

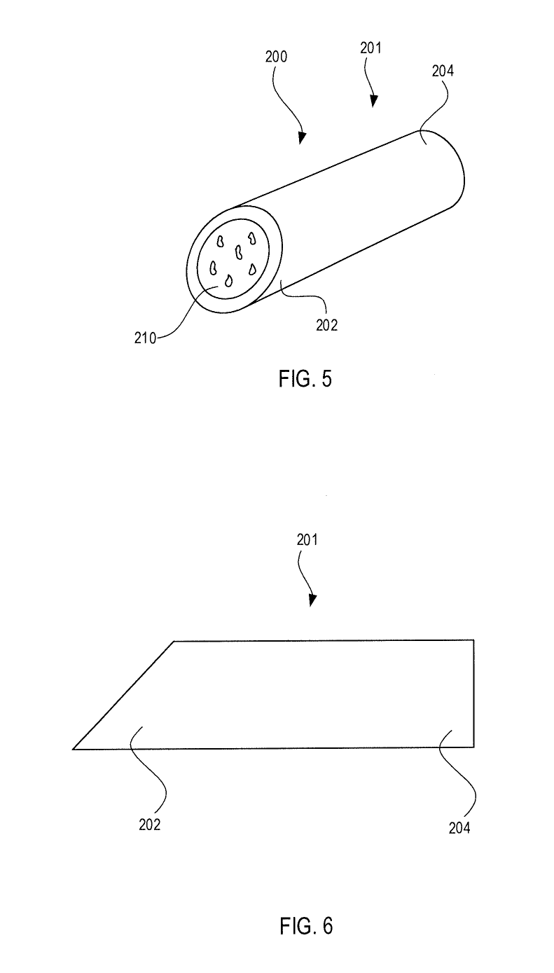

Control of rate and duration of release can also be achieved by varying the area A of the inner matrix 110 that is not covered by the outer layer 120 (i.e., the area A where the release of the active compound 105 occurs). For example, the outer layer 120 can be applied to a reduced portion of the inner matrix 110, leaving some fraction (e.g., between about 1-10% of the particle 101, etc.) uncovered (such as shown in FIGS. 8-9). In other embodiments, the uncovered end portion 102 of the particle 101 can be angled rather than perpendicular to the longitudinal axis of the particle 101, increasing the uncovered area A of the inner matrix 110 (such as is shown in FIGS. 5-6). An example of another way to modify or control the area of release, or exit or open area, is to configure the exit region such that it is reticulated or textured, which can substantially increase the surface area of the exit region.

In some embodiments, a near-zero-order (or near-constant) release can result from the particle's conformance or substantial conformance to the following conditions, the constancy or substantial constancy of which can arise from the dissolution-limited nature of the release mechanism: 1) the ratio D/(Ku) is greater than about 1, greater than about 10, or greater than about 100, 2) the ratio LK/D is less than about 0.1, or less than about 0.06, and 3) the aspect ratio L/d is between about 1 and about 50, between about 2 and about 20, or between about 2 and about 10. As previously discussed, L is the length of the particle, d is the diameter of the inner matrix, D is the diffusion constant, K the dissolution constant of the active compound 105 in the polymeric inner matrix 110, and u=1 centimeter (a standard unit of length). When considering that the ratio LK/D in the second condition is L divided by D/(Ku) when L is measured in centimeters, combining the first two conditions provides the following relationship, namely: when D/(Ku)=1, then L must be less than 0.1 cm (1 mm), and when D/(Ku)=10, then L must be less than 1 cm, and if D/(Ku) is of order 100 then any practical value of the length L is allowable.

In further embodiments, the aspect ratio L/d, where d is the diameter of the inner matrix 110, is between about 1 and about 50, between about 2 and about 20, or between about 2 and about 10. And in still further embodiments, the ratio C₀/Cₛ is at least about 5, or greater than or equal to about 10. Further, in some embodiments, if an active compound 105 (e.g., a crystalline solid active compound 105) is dispersed in a polymeric inner matrix 110 such that the following condition is satisfied: Cₛ(DK)^{1/2}˜10⁻⁹ g/(cm² sec), and Ku<D, a near-constant release over an approximate 10-year period can be achieved, (given rod-shaped or otherwise elongated particles 101 having an inner matrix 110 with a 1 mm diameter and length L on the order of 1 cm).

In certain embodiments, the ratio D/(Ku) is greater than about 1, greater than about 10, or greater than about 100. In some of such embodiments, if this ratio is greater than about 17, then, the ratio LK/D will satisfy the relation LK/D<0.06 that can be required for near-constant release, even with a rather large L of 1 cm.

As can be appreciated with regards to the discussion herein, a plot of the logarithm of the rate of release of active compound versus the logarithm of time can yield a plot (which can also be referred to as a “log-log plot” or “log-log release rate plot”) can be fit with a standard regression curve, with the slope giving the order of the release profile. Because accuracy can often be gained by plotting cumulative release amounts (or concentrations), the focus on log-log plots of cumulative release vs time, will have a slope of 1.0 in the case of a zero-order release. In some embodiments, the drug delivery systems disclosed herein yield log-log cumulative release plots with a slope that is greater than 0.75, greater than about 0.8, or greater than about 0.85. These criteria indicate that the release profile is mathematically closer to zero-order kinetics than first-order kinetics that result from most prior art drug delivery vehicles (particularly those lacking moving mechanical parts such as pistons). The nearer the slope to 1.0, the more uniform the release rate, all other things being equal. The slope should be evaluated over the entire release time-profile, including any burst effect at early time points.

In certain embodiments, if desired, an initial delay in the release profile, which could be viewed as the opposite of a burst effect, can be attained in the following way: Along with or in place of uncoated regions, portions of the inner matrix 110 could be coated by an erodible polymer such as PLGA. To the extent the erodible coating is substantially impermeable to the active compound, very little active compound would be released until the erodible regions of the coating were eroded away, after which the near-zero-order release kinetics described herein would begin. One advantage of this approach, for example, would be in the case where a ready-to-use aqueous formulation were desired but release of the active compound 105 into the aqueous medium of the formulation during the storage shelf-life were undesirable. In such instances, the erodible polymer would be selected so as to not erode significantly during storage, but would erode when placed in the body, either due to pH, enzymatic action, or the increased volume of water in the subject or patient as compared to in the vial.

FIGS. 5-6 illustrate a drug delivery system 200 according to another embodiment of the present disclosure. The system 200 can, in certain respects, resemble components of the system 100 described in connection with FIGS. 1-4 above. It will be appreciated that all the illustrated embodiments may have analogous features. Accordingly, like features are designated with like reference numerals, with the leading digits incremented to “2.” (For instance, the assembly is designated “100” in FIGS. 1-4 and an analogous assembly is designated as “200” in FIGS. 5-6.) Relevant disclosure set forth above regarding similarly identified features thus may not be repeated hereafter. Moreover, specific features of the system 200 and related components shown in FIGS. 5-6 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the system 200 of FIGS. 5-6. Any suitable combination of the features, and variations of the same, described with respect to the system 100 and components illustrated in FIGS. 1-4, can be employed with the system 200 and components of FIGS. 5-6, and vice versa. This pattern of disclosure applies equally to further embodiments depicted in subsequent figures and described hereafter.

As show in FIGS. 5-6, in some embodiments, the drug delivery system 200 includes a particle 201 having an increased area of release when compared to the particle 101 of FIGS. 1-4. For example, in FIGS. 5-6, the first end portion 202 is angled (rather than perpendicular to the longitudinal axis of the particle 201) such that the area (e.g., surface area) of the uncovered or “exposed” portion of the inner matrix 210 is increased. If desired, both ends 202, 204 can be cut or formed in an angled manner.

FIG. 7 depicts a drug delivery system 300, according to yet another embodiment of the present disclosure. As shown in FIG. 7, in some embodiments, the uncovered or “exposed” end 302 of the inner matrix (not shown), where the active compound (not shown) is configured to egress from the particle 301, can be covered by a semipermeable membrane 330 that permits passage of the active compound (not shown), for example, in aqueous solution with the water hydrating the membrane 330, but impedes the ingress of oils and/or lipids. The membrane 330 can include various materials, including, but not limited to, polymers or copolymers of polyvinylidene fluoride (“PVDF”), polyethersulfone (“PES”), hydrophilic cellulose-derived membranes, polyacrylonitrile (“PAN”), hydrophilic polycarbonates, polyvinyl alcohol (“PVA”), hydrophilic nylons, and derivatives and mixtures thereof. Hydrophilic membranes 330 can also be used that are optimized for minimizing adsorption of proteinaceous material.

FIGS. 8-9 depict a drug delivery system 400, according to another embodiment of the present disclosure. As shown in FIGS. 8-9, in some embodiments, a length or a portion of the inner matrix 410 can be left uncoated, or patterned-uncoated. For example, one or both end portions 402, 404 of the particle 401 can be left uncoated as shown in the illustrated embodiment. In such embodiments, the area of the inner matrix 410 that is uncovered can be substantially increased (e.g., 10%, 20%, 30%, 40%, 50%, etc. or more surface area). In certain embodiments, the fraction of total area of particle 401, including the circumferential area as well as that of the two end faces, that is uncoated can be less than about 20%, less than about 10%, or less than or equal to about 5% by area of the inner matrix 410.

As can be appreciated, while much of the present disclosure is related to drug delivery systems that includes particles, other types of drug delivery systems can also be used. For example, in some embodiments, the drug delivery system includes a surgical suture. Like the particles disclosed above, the suture can include an outer layer and an inner matrix that includes a biologically active compound. The outer layer can provide adequate tensile strength and be flexible, which can be required of the suture. One or more non-covered regions could be spaced along the suture, for example, at regular intervals along the longitudinal length of the suture.

The multilayered polymer arrangements described herein can also be produced by various methods known in the art, such as extrusion or co-extrusion processes, or by molding (e.g., injection molding) or similar process. For example, a powdered form of the active compound, obtained by wet or dry milling, controlled precipitation, spray-drying, etc., of the desired crystal size distribution, may be first mixed into the material used to form the inner matrix, with elevated temperature if required to soften the polymer. If a solvent-free method is used, then preferably the matrix polymer is either uncrosslinked at this point, or only lightly crosslinked; further crosslinking, if desired, can be applied at any stage subsequent to this mixing, and may even be engineered to occur during the mixing in a single operation (e.g., due to the elevation in temperature). While standard processes of intensive mixing, kneading, or alternatively convective mixing or homogenizing (e.g., at elevated temperatures), and the like can be applied. An alternative is melt-blowing with an impacting stream of the powder, thus creating fiber contemporaneously with powder/polymer mixing. The matrix/active dispersion (which may at elevated temperatures in fact be a solid-in-liquid dispersion, or even an emulsion if the melting point of the active is low), is then extruded into the desired shape, typically a fiber, and the outer layer, (coating or skin) can be applied either concomitantly using co-extrusion, or to the extruded fiber using standard methods of coating, such as spray coating, spray-drying, electrospray, fluidized bed coating, vapor deposition, etc. Roll-coating processes might be advantageous if the fiber is produced as a (woven or non-woven) web, which after coating would be subsequently broken or cut into segments of the desired length.

Methods of using the drug delivery systems are also disclosed herein. In particular, it is contemplated that any of the components, principles, and/or embodiments discussed above may be utilized in either a drug delivery system or a method of using the same. For example, in an embodiment, a method of delivering a biologically active compound to a mammal can include obtaining a biocompatible particle comprising a polymeric inner matrix comprising a biologically active compound; and a polymeric outer layer disposed at least partially around the inner matrix, and injecting or implanting the particle into a body of a mammal. The method can further include a step of explanting the particle, for example, after the active compound has been exhausted or substantially exhausted from the particle. In other embodiments, the particle can be configured to be left within the body of the mammal. Additional steps, and/or methods, can also be employed.

### Examples

The following examples are illustrative of embodiments of the present disclosure, as described above, and are not meant to be limiting in any way.

### Example 1

A thermoplastic food-grade elastomer was obtained from Shenzhen Zhongsuwang Plastic Products Co., Ltd. (TC-75AN grade, food grade copolymer containing styrene/ethylene/butylene/styrene (“SEBS”)). 0.343 g of the elastomer was heated to melt, and 0.046 g of usnic acid (an active compound) was added, resulting in a mixture of about 11.8% by weight of usnic acid. The mixture was then formed into a cylindrical or rod-shaped inner matrix.

The cylindrical or rod-shaped inner matrix, having a weight of about 33 mg, was loaded into a 2-inch length of polyolefin heat-shrink tubing, Thermo-Sleeve HST332BK100 with a 3/32″ diameter and a 2:1 maximum shrink ratio. Based on the 11.8% by weight concentration of usnic acid in the inner matrix mixture, the inner matrix contained approximately 3.9 mg of usnic acid. The tubing was heated mildly by holding it above, but not in contact with, a hot plate, resulting in shrinkage of the tubing and establishing a tight fit between the tubing and the inner matrix. One end of this tubing, over which the polymer/usnic mixture was flush with the end of the tubing, making the diameter of the uncovered region an approximate circle of diameter 2 mm, and length just over 1 cm.

The uncovered end of the particle was immersed in a C18E80/t-BA/water receiving medium, containing 0.6% tert-butyl acetate and 0.1% of the nonionic surfactant octadecyl-poly(oxyethylene)₃₀, which has a polar group of approximately 80 oxyethylene units. Absorbance measurements were then obtained from a Pharmacia Ultrospec 3000 spectrometer at 290 nm, using the receiving medium (without any exposure to usnic acid) as the reference liquid. Quartz cuvettes of 1 cm width were used for the absorbance measurements. Usnic acid is known to have an absorbance peak near 282 nm with an extinction coefficient on the order of 20,000 M⁻¹. The concentration of usnic acid at saturation was approximately 44 mg/L, or 0.130 mM, and at this concentration the absorbance at 290 nm was found to be approximately 3.20.

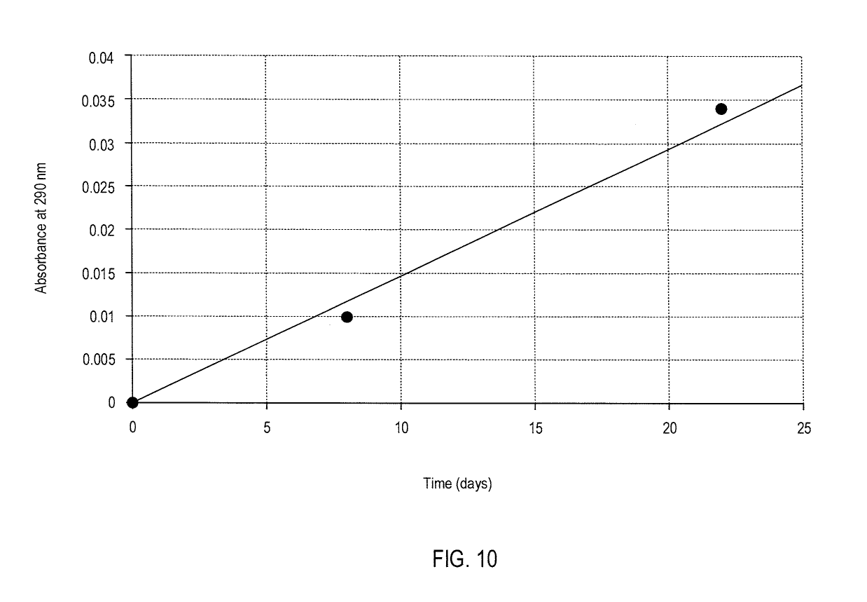

Eight days after placing the particle in the receiving medium, the absorbance was measured to be 0.010 Absorbance units; at the 22-day point the absorbance was 0.034. The plot of FIG. 10 shows the linearity to this release.

### Example 2

Samples of rod-like particles were prepared having the following characteristics: Coated length L was approximately 50 mm, with approximately 3 mm uncoated at each end of each coated length, and the diameter of the inner matrix was approximately 0.2 to 0.3 mm; this diameter included a contribution from a support substrate that was a 150 Denier polyester yarn. For purposes of illustrating the release profile of these rod-like particles, the present test was performed using conceptual rod-like particles having an inner matrix diameter of approximately 0.2 to 0.3 mm, length L of about 5 cm, and open regions or end portions 402 (as illustrated in FIG. 8) of about 3 mm, that are configured such that the longitudinal axes of the rod-like particles form a cumulative length of about one yard (i.e., using a polyester, multifilament, texturized yarn of 150 Denier as a substrate for the inner polymeric matrix). Terbinafine hydrochloride (an active compound), as a fine powder, was incorporated into the inner matrix at approximately 10 wt %. Five inner matrix polymers with elastomeric properties were investigated and each coated with two outer “coating” polymers, and a third sample left uncoated as a control.

Inner Matrix Polymers: “AlberdingkUSA U 3700 VP”, identified in the table below as “3700”, is an aqueous dispersion of an aliphatic polycarbonate-polyurethane without free isocyanate groups. “Rovene 4021”, identified in the table below as “4021”, is a self-crosslinking, carboxylated, poly(styrene-b-butadiene) block copolymer from Mallard Creek Polymers; it is an aqueous dispersion at 50% solids; the elastomer (butadiene) content is 33% of the block copolymer. “AlberdingkUSA M 2065”, identified in the table below as “2065”, is an aqueous dispersion (50% solids) of a styrene-acrylic copolymer with a Tg of about −24 degrees C. Novagard 200-260 silicone RTV, identified in the table below as “RTV”, is a 100% solids, low-viscosity silicone formulation made by Novagard that crosslinks via an oxime-based mechanism upon exposure to humidity or moisture. “AlberdingkUSA AC 2310”, identified in the table below as “2310”, is an aqueous dispersion of an acrylic polymer which upon curing has a T_{g} of −45 degrees C.

Coating Polymers: “ZAR Exterior Polyurethane”, identified in the table below as “ZAR”, is a water-based coating that is recommended for marine applications, made by United Gilsonite Laboratories. “AlberdingkUSA U 933”, identified in the table below as “U933”, is an aliphatic polycarbonate-polyurethane aqueous dispersion; it is about 35% polymer.

After the inner matrix polymer cured, the coating applied, and the coating allowed to dry, the samples were washed with water (exposure time to water approximately 4 seconds), and then immersed in 20 mL of distilled water and placed on a laboratory rocker that gently rocked the capped vials. For measurement of released drug, each sample was overturned to gently mix the contents, and a quartz cuvette filled with liquid from the vial; absorbance was then measured at 273 nm.

After 24 hours, a measurement was taken to evaluate the effectiveness of the coating at limiting the release of active (terbinafine). The lower the ratio of terbinafine released from a given coated sample to that released from the uncoated control, indicated as “ABS/control” in the table below, the better is the performance of the coating on that matrix. It should be noted that the matrix is not fully coated, since these are embodiments of the disclosure and have uncoated regions for release of the active compound, and so the ratio of ABS/control should not equal zero.

| Inner Matrix Polymer | Coating Polymer | ABS/Control |
|---|---|---|
| 3700 | U 933 | 0.281 |
| 3700 | ZAR | 0.363 |
| 3700 | control | 1.000 |
| 2065 | U 933 | 0.469 |
| 2065 | ZAR | 0.181 |
| 2065 | control | 1.000 |
| RTV | U 933 | 0.717 |
| RTV | ZAR | 0.248 |
| RTV | control | 1.000 |
| 2310 | U 933 | 0.881 |
| 2310 | ZAR | 0.318 |
| 2310 | control | 1.000 |
| 4021 | U 933 | 0.039 |
| 4021 | ZAR | 0.058 |
| 4021 | control | 1.000 |

The three “4021” samples were followed for a week, and the table below shows the absorbances at 273 nm, which in view of very small absorbances for placebo samples (having no active), are very nearly linear with concentration, a very close approximation that was also borne out by calibration curve measurements.

A table showing UV-Vis spectrometry measurements at 273 nm for the samples loaded with terbinafine hydrochloride, releasing into distilled water, with Rovene 4021 as the inner matrix polymer in which the terbinafine HCl is dispersed, and where the outer layer is either “ZAR” polyurethane, AlberdingkUSA U 933, or no coating (control) is shown below:

| | Outer | ABS 273 | ABS 273 | ABS 273 |
|---|---|---|---|---|
| Sample | Layer | (After 1 Day 1) | (After 2 Days) | (After 3 Days) |
| 1 | U 933 | 0.08 | 0.077 | 0.065 |
| 2 | ZAR | 0.12 | 0.137 | 0.317 |
| 3 | Control | 2.074 | 2.522 | 3.08 |

The first row (Sample 1) shows that the active compound (terbinafine hydrochloride) dispersed in the inner polymer (“Rovene 4021”) matrix, and partially coated with AlberdingkUSA U 933, released the active compound too slowly to be meaningfully measured at the 1-week point. The second row (Sample 2) shows that the active compound (terbinafine hydrochloride), again dispersed in the inner polymer (Rovene 4021) matrix, and partially coated (approximately 90% of the length coated) with ZAR polyurethane from United Gilsonite, releases the active compound into water at the rate of about 10% of the active compound in one week; extrapolation of these one-week results indicates that the duration of terbinafine release is likely on the order of 10 weeks, based on 10% active compound released at the one-week point.

The Rovene 4021 matrix yielding the slowest release in this experiment may be related to a potential electrostatic matrix-drug attractive interaction. The “4021” formulation features carboxylic groups as part of the basis polymer, and these can ion-pair with terbinafine, which is a basic compound.

### Example 3

As in Example 2, a powderized active compound—dantrolene sodium—was dispersed in the inner matrix material (at approx. 5 wt % loading), which was composed of Room Temperature Vulcanized (RTV) silicone marketed as Novagard 200-260. The inner matrix was then cured via a humidity-triggered oxime-based crosslinking reaction. The “ZAR” polyurethane and “U 933” outer layers were thereafter applied to control the release from the RTV inner matrix. The coated length L of the rod-like segments was approximately 50 mm, with approximately 3 mm uncoated at each end of each coated segment, and the diameter of the inner matrix was approximately 0.2 to 0.3 mm.

Dantrolene sodium is an orange-colored compound, which also forms an orange-colored solution in water above about pH 9.5. Absorbances were measured at 380 nm. For purposes of illustrating the release profile of these rod-like particles, the present test was performed using conceptual rod-like particles having an inner matrix diameter of approximately 0.2 to 0.3 mm, length L of about 5 cm, and open regions or end portions 402 (as illustrated in FIG. 8) of about 3 mm, that are configured such that the longitudinal axes of the rod-like particles form a cumulative length of about one yard. This configuration of the rod-like particles (in the form of a cumulative length of about one yard) was placed in about 20 mL of aqueous buffer at pH about 11, and ABS380 measured at 7 days, 19 days, and 21 days:

| | Inner | Outer | ABS 380 | ABS 380 | ABS 380 |
|---|---|---|---|---|---|
| Sample | Matrix | Layer | (at 7 Days) | (at 19 Days) | (at 21 Days) |
| 1 | RTV | U 933 | 0.030 | 0.069 | 0.072 |
| 2 | RTV | ZAR | 0.041 | 0.103 | 0.117 |
| 3 | RTV | Control | 0.320 | 0.563 | 0.594 |

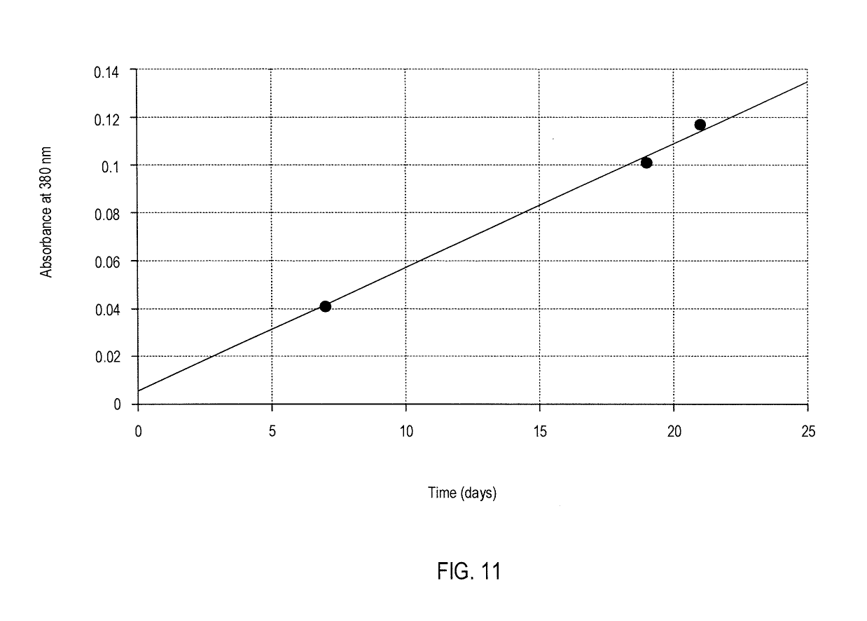

As seen from the table above, the ZAR and 933 partial-coatings reduced the first-week release of dantrolene by an order of magnitude. Furthermore, the release from the U 933 coated sample was near-zero order with a slope of approximately 0.035 Absorbance units per day. And the ZAR-coated sample also exhibited a very nearly constant release rate, i.e., near-zero order release, yielding 0.0057 Absorbance units per day which is illustrated in the plot of FIG. 11. Dantrolene sodium, being strongly-colored allowing Absorbances to be measured in the visible range which is less susceptible to interference than the UV range, and possessing a modest aqueous solubility, is very nearly an optimal marker in this measurement; indeed, visibly one can observe the color very gradually and reliably move from the particle to the aqueous release medium. The plot in FIG. 11 includes a least-squares fit line, and the extrapolation back to zero time yields a very small number thus indicating the absence of any “burst”, as will be recognized by one skilled in the art. With the uncoated control apparently levelling out at an ABS 380 of about 0.6, and the best-fit line reaching an absorbance of 0.1 at approximately the 3-week point, this extrapolates to a release over about 18 weeks. Slow, near-zero-order release of dantrolene in, e.g., a region of skeletomuscular damage can provide strong anti-spasmotic activity with pain-relieving action that could potentially reduce the need for systemic administration of narcotic drugs.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. The scope of the invention is therefore defined by the following claims and their equivalents.

What is claimed is:

1. A drug delivery system, comprising:
an implantable or injectable biocompatible particle, comprising:
a polymeric inner matrix comprising a biologically active compound; and
a polymeric outer layer disposed at least partially around the inner matrix, the polymeric outer layer being substantially impermeable to the biologically active compound,
wherein the particle is configured to exhibit zero-order or near-zero-order release of the biologically active compound.

2. The drug delivery system of claim 1, wherein the particle is configured to exhibit a release profile which on a log-log plot of cumulative release versus time has a slope greater than or equal to about 0.62, greater than or equal to about 0.75, or greater than or equal to about 0.87.

3. The drug delivery system of claim 1, wherein the particle conforms to the following mathematical conditions: the ratio D/(Ku) is greater than about 1, the ratio LK/D is less than about 0.1, and the aspect ratio L/d is between about 1 and about 50, where L is the length of the particle, d is the diameter of the inner matrix, D is the diffusion constant, and K is the dissolution constant of the active compound in the inner matrix, and u=1 cm.

4. The drug delivery system of claim 1, wherein the polymeric inner matrix is biodegradable.

5. The drug delivery system of claim 1, wherein the polymeric inner matrix is non-biodegradable.

6. The drug delivery system of claim 1, wherein the particle is cylindrical or rod-like in shape.

7. The drug delivery system of claim 1, wherein the polymeric inner matrix comprises one or more fluoroelastomers or fluorogreases.

8. The drug delivery system of claim 1, wherein the particle is void of any mechanically movable parts.

9. The drug delivery system of claim 1, wherein the active compound comprises one or more anti-abuse or “replacement therapy” drugs, local anesthetics, opioids, steroid, peptide hormones, insulin sensitizers, multiple sclerosis related drugs, anticancer drugs, statins, TNF inhibitors, cannabinoids, migraine related drugs, vasodilators, anticonvulsants, weight loss agents, gastrointestinal (“GI”) tract drugs, cardiac drugs, anti-HIV drugs, psychiatric drugs, systemic drugs, ophthalmic related drugs, or glaucoma related drugs.

10. The drug delivery system of claim 1, wherein the particle further comprises at least one coating disposed on an exterior surface of the polymeric outer layer.

11. A method of delivering a biologically active compound to a mammal, comprising:
obtaining a biocompatible particle, comprising:
a polymeric inner matrix comprising a biologically active compound; and
a polymeric outer layer disposed at least partially around the inner matrix, and
injecting or implanting the particle into a body of a mammal,
wherein the particle is configured to exhibit zero-order or near-zero-order release of the biologically active compound.

12. The method of claim 11, further comprising:
explanting the particle after the active compound has been exhausted from the particle.

13. The method of claim 11, wherein the particle is configured to be left within the body of the mammal.

14. The method of claim 11, wherein injecting or implanting the particle comprises subcutaneous, intramuscular, intradermal, or intraocular injection or implantation.

15. The method of claim 11, wherein the method is used in the treatment of a cancer.

16. The method of claim 15, wherein the cancer is selected from at least one of bone cancer and breast cancer.

17. The method of claim 11, wherein the particle comprises one or more materials that promote the ingrowth of tissue onto the particle.

18. The method of claim 11, wherein the polymeric inner matrix comprises one or more fluoroelastomers or fluorogreases.

19. The method of claim 11, wherein the polymeric inner matrix is biodegradable.

20. The method of claim 11, wherein the polymeric inner matrix is non-biodegradable.
